(19) United States
(12) Patent Application Publication
(10) Pub. No.: US 2010/0055787 A1
(43) Pub. Date: Mar. 4, 2010

(54) IN-VITRO PEARL PRODUCTION USING MARINE ORGANISMS

(75) Inventors: Seeni Naicker Dharmaraj, Tuticorin, IN (US); Paulose Suja Cheruvathoor, Tuticorin, IN (US)

Correspondence Address: HARNESS, DICKEY, & PIERCE, P.L.C, 7700 Bonhomme, Suite 400, ST. LOUIS, MO, 63105, US

(73) Assignee: Indian Council of Agricultural Research, New Delhi (IN)

(21) Appl. No.: 12/515,892

(22) PCT Filed: Nov. 22, 2006
(86) PCT No.: PCT/IB06/03299

§ 371 Date: Jun. 3, 2009

(51) Int. Cl. C12N 5/00

(52) U.S. Cl. 435/375

(57) ABSTRACT

This invention in general relates to the production of pearls using marine organisms. More particularly, the present invention relates to in-vitro pearl production wherein shape, size and colour of the pearl can be controlled. A semi solid medium is used in the present invention which facilitates the bead to stand stationary and the cells grow over and around the bead and nacre is deposited within 15 days of initiation of cultures. Shell beads treated in organ cultures of mantle tissue of pearl oyster Pinctada fucata were harvested at different intervals and Scanning Electron Microscopy (SEM) shows that nacre growth continued to grow up to 10 months. Similarly the nacre growth was tested in the organ cultures of mantle tissue of abalone Haliotis varia, and the results indicated that there was continuous growth of nacre throughout and all the shell beads got nacre coating.

### FIELD OF THE PRESENT INVENTION

This invention relates to an in vitro process for pearl production. More particularly the present invention relates to in vitro pearl production using marine organism.

### BACKGROUND AND PRIOR ART

A pearl is produced in a live pearl oyster which is totally dependent on the natural environment. Any change in the environment would affect the entire population of pearl oyster which is very sensitive to any change. According to publication of Aquaculture magazine of September 1991, the world's supply of natural pearls( both fresh water and sea water) was reaching the point of exhaustion, if it were not for the invention of the “cultured” pearl, there would be no pearl industry today, and the value of a natural pearl necklace would be astronomical.

Further to this, all existing in vivo processes for pearl production inherently carry various draw backs, such as uncertainty of survival of pearl oyster in the natural environment, no guarantee for quality pearls, no chance of manipulation of pearl production process, excessive cost of production and difficulty in placing a bead in the mantle of organism etc.

More over, survival of nucleated oysters in the natural habitat is not guaranteed, as marine pollution is increasing at global level. Production of free and spherical pearls is not practically feasible in the case of abalone because of the powerful movement of its foot. However production of semi-spherical pearls has been demonstrated by fixing shell bead nucleus between the mantle and shell of abalone. The technology for the production of semi-spherical pearls has its own limitations as the pearls produced have to be removed forcibly from the shell and have to be processed. It is an impediment to large scale production of semi-spherical pearls. Hence, the situation warranted an alternative way to overcome the uncertainty and the outcome is the method of production of pearls through tissue culture, which totally avoids the dependence of natural environment for the production of pearls and facilitates the production of free coloured pearls from the abalone.

Wada (1961) found, during shell formation and regeneration of the pearl oyster, Pinctada fucata, induction of nucleation from the sub microscopic nuclei for the development of crystals and reported the growth of crystals and subsequent formation of nacreous layer.

Machii (1974) performed organ culture of the mantle tissue of the pearl oyster, Pinctada fucata (Gould) by plasma clot method using glass petri dish. In the culture roundish epithelial-like cells, pigmented epithelial-like cells, spindle shaped muscle cell or string-like muscle bund were emerged from the mantle tissue. After 12 days of initiation of culture, depositions of organic substances occurred from regenerated epithelia.

Machii and Wada (1989) organised explant cultures of mantle tissue of the pearl oyster, Pinctada fucata where discolouration of explant tissue occurred. Secretion of organic substance was recorded on the surface of the explant. After longer maintenance of this culture resulted in trefoil-like depositions at the margin of the explant itself. The shape of such depositions resembles prismatic crystals formed in-vivo at the initiation of pearl formation and shell regeneration.

Samata et al., (1994) conducted experiments on the culture of mantle tissue of the pearl oyster Pinctada fucata and noticed a black secretion on the explant after 15 to 30 days of initiation of culture. The black secretion induced the crystal growth. When the crystals were subjected to analysis through the Scanning Electron Microscopy (SEM) and through the Energy Dispersive X-Ray Microanalyser (EDS) they revealed the presence of large amounts of calcium and sulpher.

Uno, as early as in 1957, produced semi-spherical pearls in the abalones such as Haliotis discus, H. gigantea and Hseiboldii. Fankboner (1991, 1993) implanted nucleus in the pinto abalone Haliotis kamatschatkana and produced gem quality marine pearls in North America. Fankboner (1994 a and 1994b) has further experimented with producing bead-nucleated pearls and claimed to have cultured a few whole free formed tissue-nucleated pearls as large as 8 mm. Victor et al., (2001) reported experimental production of half pearls in the tropical abalone Haliotis varia in India.

Current Science, 86 (5): 730-734 discloses that for fresh water tissue culture complete nacre layer formation is not achieved and they have followed the open type incubation in Petri plates. Rinkevich, R. 1999. J. Biotech., 70(1-3): 133-153 reveals cell culture in marine invertebrates. (page 139, line 8-19: Studies of mantle tissue culture from the pearl oyster are similarly disappointing. Pearl oyster cell culture started almost 25 years ago (Machii, 1974) with tissue explants. In this culture system, secretion of an organic substance was followed in-vitro. A year later, the same laboratory documented mitoses in cell cultures from mantle tissue. Even with the importance of this industry of pearl oysters, recent studies (Awaji, 1991, 1997; and literature therein) do not show any significant improvement as compared to the 1970s cell culture studies; page 140, line 14-24: There are still difficulties common to primary molluscan cultures, such as tissue sterilization, cell proliferation, cellular purification/characerization and thraustochytrid contamination (part of this list is ummarized in Awaji (1997). The above studies embraced the four culture situations uniquely characteristic of a variety marine molluscs: giant neurons routinely used n neurobiology (Tamse et al 1995),pearl formation (Awaji 1991, 1997 and literature there in: Samata et al, 1994). This review analyzes activities in the field of marine invertebrate cell culture during the years 1999 to 2004 and compares the outcomes with those of the preceding decade (1988 to 1998). During the last 5 years, 90 reports of primary cell culture studies of marine organisms belonging to only 6 taxa (Porifera, Cnidaria, Crustacea, Mollusca, Echinodermata, and Urochordata) have been published. In recent years studies attempting to improve cell culture methodologies have decreased, while interest in applications of already existing methodologies has increased. This reflects the effects of short-term cultures in attracting new researchers and scientific disciplines to the field. Second, only 17.8% of the recent publications used long-term cultures, compared with 30.0% of the publications in the previous decade. Third, during recent years research in cell cultures has studied fewer model species more extensively (mainly, Botryllus schlosseri, Crassostrea, Mytilus, Penaeus, and Suberites domuncula), signifying a shift from previous investigations that had studied a more diverse range of organisms. Still, not even a single established cell line from any marine invertebrate has yet been made available. However, the use of new cellular, genomic, and proteomic tools may fundamentally change our strategy for the development of cell cultures from marine invertebrates.

More over, the existing processes failed to produce a layer over the bead Because the bead in the liquid medium will roll and it is not possible for the cells to grow around and produce the nacre layer, to form an in-vitro pearl.

In order to overcome the above said technical problem, the present invention involves semi solid medium which facilitates the bead to stand stationary and the cells grow over and around the bead and a complete nacre layer formed.

At the outset of the description which follows it is to be understood that ensuring description only illustrates a particular form of this invention. However, such particular form is only an exemplary embodiment without intending to imply any limitation on the scope of this invention. Accordingly, the description is to be understood as an exemplary embodiment and reading of the invention is not intended to be taken restrictively.

### OBJECTIVES OF THE PRESENT INVENTION

It is the primary object of invention to develop an in-vitro pearl production using marine organisms. Further, in another object of the present invention is to develop a semisolid medium for pearl production so as to reduce the movement of the beads. Yet in another object of the present invention is to optimize various process parameters to reduce the nacre formation time. One more, object of the present invention is to develop a mechanism to control the shape, size and colour of the pearl.

### SUMMARY OF THE PRESENT INVENTION

This invention in general relates to the production of pearls. In particular this invention relates to novel method of production of in-vitro pearls through tissue culture. Semi solid medium is used in the present invention which facilitates the bead to stand stationary and the cells grow over and around the bead and nacre is deposited within 15 days of initiation of cultures.

Shell beads treated in organ cultures of mantle tissue of pearl oyster Pinctada fucata were harvested at different intervals and Scanning Electron Microscopy shows that nacre growth continued to grow up to 10 months. Similarly the nacre growth was tested in the organ cultures of mantle tissue of abalone Haliotis varia and the results indicated that there was continuous growth of nacre throughout and all the shell beads got nacre coating.

### BRIEF DESCRIPTION OF THE DRAWINGS

FIG. 1. A method of placement of shell beads

FIG. 2: Shell bead nucleus with explant in an organ culture of pearl oyster mantle

FIG. 3: Deposition of crystals over the bead in pearl oyster

FIG. 4: Formation of nacreous layer and organic matrix in pearl oyster

FIG. 5: Shell bead nucleus with explant in an organ culture of abalone mantle

FIG. 6: Deposition of crystals over the bead in abalone

FIG. 7: Formation of nacreous layer and organic matrix in abalone

FIG. 8: Growth of nacre at three months in Pinctada fucata

FIG. 9: Growth of nacre at six months in Pinctada fucata

FIG. 10: Growth of nacre at nine months in Pinctada fucata

FIG. 11: Growth of nacre at ten months in Pinctada fucata

FIG. 12: Growth of nacre at three months in Haliotis varia

FIG. 13: Growth of nacre at six months in Haliotis varia

FIG. 14: Growth of nacre at nine months in Haliotis varia

FIG. 15: Growth of nacre at fourteen months in Haliotis varia

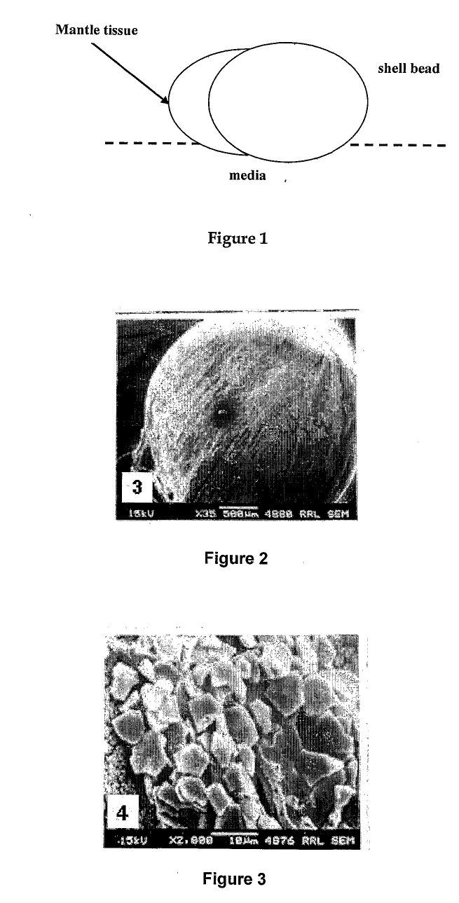

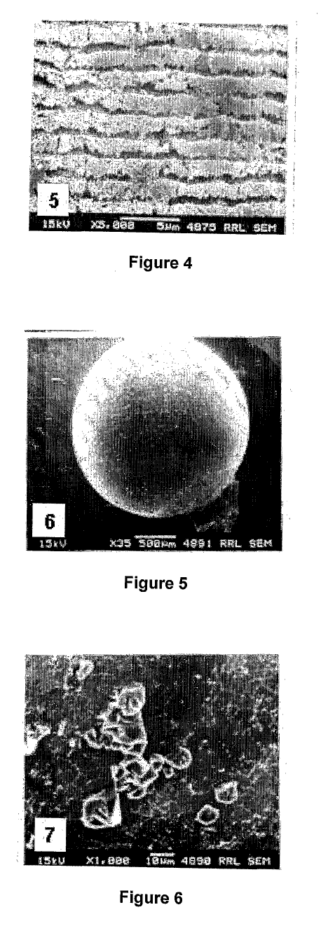

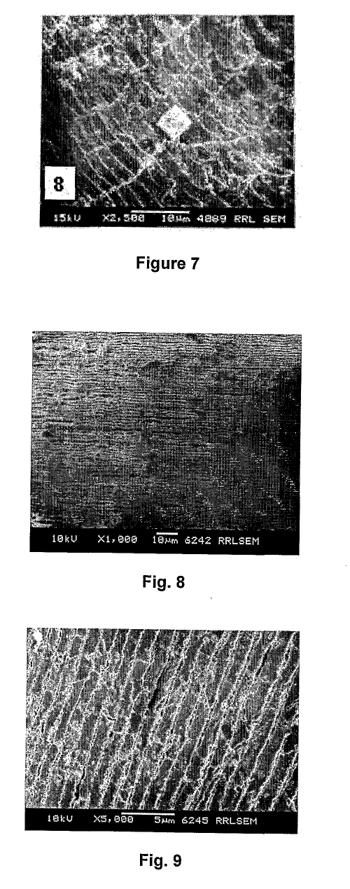

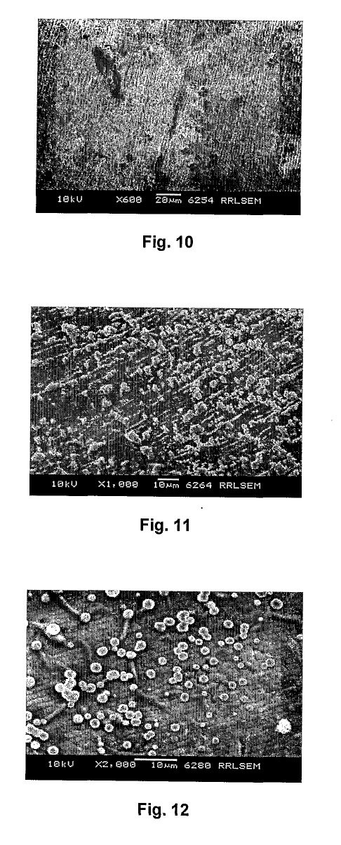

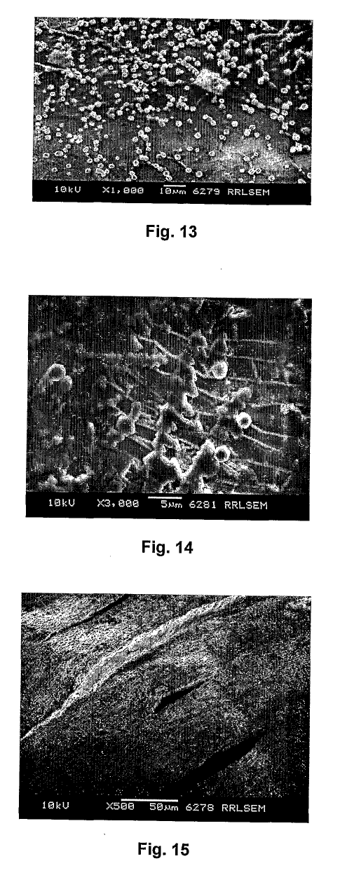

### DETAILED DESCRIPTION OF THE INVENTION

This invention in general relates to the production of pearls. More particularly, the present invention relates to in vitro pearl production using marine organism The pearl oysters (Pinctada fucata) and the abalones (Haliotis varia), prior to experimentation, are depurated in U.V. irradiated running seawater for 3 days. The outer shell surface of these depurated oysters is wiped with 70% alcohol and the oysters are opened by a sterile knife. Mantle tissue is excised from both shell valves. Prior to cleaning, the pallial organs on the outer free margin and the inner connective tissue of the mantle are removed by cutting with sterile knife. The selected mantle strip is washed thoroughly in sterile natural seawater till the mucus and other adhering particles are removed fully. The strip is then treated in antibiotic solution containing streptomycin and penicillin. Further washing in sterile seawater is continued to remove the effect of antibiotic solution. The processed strip is cut in to pieces of 2 mm square in the clean bench. Each piece of the mantle along with a sterile shell bead is placed in a culture flask containing nutrient rich medium. The round epithelial-like cells proliferated from the mantle tissue and spread over the shell bead. Accumulation of such cells on the shell bead resulted in the formation of pearl sac. Later the cells of the pearl sac produced organic matrix. The organic matrix induces the cells to secrete crystals and deposit on the bead. The crystals develop from sub microscopic nuclei that form by a process known as nucleation. The organic matrix provides nucleation sites for crystal formation and influences the type of crystals to be produced. Nacre is deposited within 15 days of initiation of cultures. Addition of more such crystals resulted in the formation of nacreous layer. By the formation of such nacreous layer an in-vitro pearl is formed. Formation of organic matrix and nacreous layer has been clearly demonstrated using a tiny piece of mantle tissue of the pearl oyster, Pinctada fucata and of abalone (Haliotis varia) at laboratory conditions and the results were confirmed by the Scanning Electron Microscopy (SEM) and Energy Dispersive X-ray micro analyser (EDS) in both cases.

### Flow Chart Showing in-vitro Method for Pearl Production in Marine Molluscs

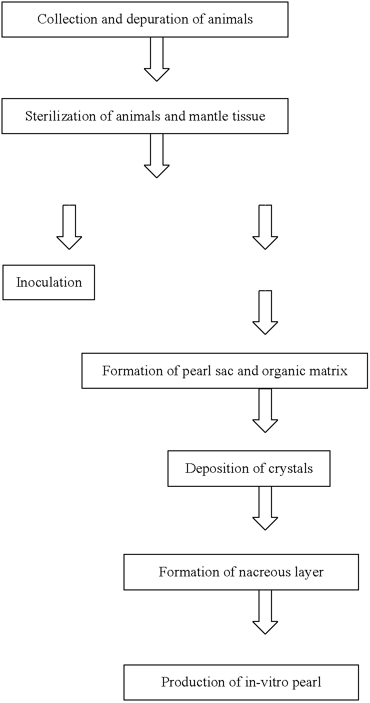

Having achieved a success in the basic technology in in-vitro pearl production wherein the formation of nacreous layer was demonstrated, further experiments were conducted to study whether the nacre growth is continuously growing throughout the culture period or the growth is ceased at a particular period of time in in-vitro conditions. Specific experiment was conducted in the above manner in both species with sterile spherical shell beads to assess the nacre growth at particular intervals. Shell beads treated in organ cultures of mantle tissue of pearl oyster Pinctada fucata were harvested at different intervals namely 3, 6, 9 and 10 months. The shell beads were analysed in

Scanning Electron Microscopy and found that the nacre growth continued to grow up to 10 months.

Similarly the nacre growth was tested in the organ cultures of mantle tissue of abalone Haliotis varia. The shell beads of these cultures were harvested at different intervals namely 3, 6, 9, and 14 months. The nacre growth during these periods was assessed in SEM. The results indicated that there was continuous growth of nacre throughout and all the shell beads got nacre coating.

For experimental purposes and for our convenience spherical shell beads are currently used. Apart from this, any shape (square, elliptical etc.) can be used in this method. Size of shell beads ranging 2 mm to more than 8 mm can easily be accommodated in our culture systems. The technology provides ample chances for production of desired coloured pearls by the manipulation of the composition of media.

### Composition of Nutrient Rich Medium

(a) 0.8 gm agar powder dissolved in 57.2 ml triple distilled water

(b) Supplementary salt solution consisting of:

| | | |
|---|---|---|
| NaCl | 102.4 | g/l |
| KCl | 1.8 | g/l |
| CaCl₂ | 1 | g/l |
| MgCl₂ | 8 | g/l |
| MgSO₄ | 16.7 | g/l |
| M 199 10x | 2 | ml |
| FCS | 10 | ml |
| Lactalbumin | 5 | ml |
| hydrolysate(10%) | | |
| NaHCO₃ (5%) | 0.7 | ml |
| Kanamycin(0.1 g/ml) | 1 | ml |

### Examples

### Example 1

### Placement of Shell Beads

The present invention uses semisolid culture medium which prevents rolling of shell bead. After placing the sterile beads inside the media, the mantle piece is attached to one side of the bead so that one side of the tissue is touching the medium.

### Example 2

Prior to organization of cultures the pearl oysters were depurated 2 to 5 days and surface is sterilized with 60 to 80% v/v alcohol. The mantle tissues excised from the depurated animals were processed by washing in sterile sea water and sterilized by treating in antibiotic solution in specific doses of 500 to 1500 μg/ml streptomycin and 1000 to 3000 IU/ml penicillin.

Following the above procedures a series of experiments were conducted on organ cultures of mantle tissue of pearl oyster in which sterile shell beads attached with tissue explants were placed in nutrient rich culture medium in culture flasks. These flasks were incubated in temperature of 25 to 30° C.. Formation of pearl sac and organic matrix; secretion of crystals by the cells and deposition of the same on the organic matrix and formation of nacreous layer were successfully achieved in the species. The shell beads treated in organ cultures of the species were collected after 10 to 14 months of culture and later analyzed at a time for their surface structure through Scanning Electron Microscope (SEM). The results indicated that the structure and the pattern of development of nacreous layer were found to be species specific.

### Example 3

Prior to organization of cultures the abalones were depurated 2 to 5 days and surface sterilized with 60 to 80% v/v alcohol. The mantle tissues excised from the depurated animals were processed by washing in sterile sea water and sterilized by treating in antibiotic solution in specific doses of 500 to 1500 μg/ml streptomycin and 1000 to 3000 IU/ml penicillin.

Following the above procedures a series of experiments were conducted on organ cultures of mantle tissue of abalone in which sterile shell beads attached with tissue explants were placed in nutrient rich culture medium in culture flasks. These flasks were incubated in temperature of 25 to 30° C.. Formation of pearl sac and organic matrix; secretion of crystals by the cells and deposition of the same on the organic matrix and formation of nacreous layer were successfully achieved in the species. The shell beads treated in organ cultures of the species were collected after 10 to 14 months of culture and later analyzed at a time for their surface structure through Scanning Electron Microscope (SEM). The results indicated that the structure and the pattern of development of nacreous layer were found to be species specific.

### Example 4

Pearls of different sizes are obtained by using different size of shell beads even up to 8 mm with larger piece of mantle tissue. Instead of round shell bead, beads of different shape are placed in the medium using the same procedure for obtaining pearls of different shapes. Coloured pearls are obtained by the same procedure incorporating different metals like manganese, copper, zinc and iron in the same medium.

### Advantages of the Present Invention

1. Under conventional methods, three pearls can be produced in an oyster in its lifetime, whereas in tissue culture methods a mantle piece of 2 mm square is capable of producing an in-vitro pearl. A live oyster can yield numerous mantle pieces and therefore more number of pearls can be produced.
2. Production of free pearls in abalones is feasible in this technology.
3. Large sized pearls may be produced.
4. Pearls of different sizes, shapes and colours are produced.
5. Semi solid medium is used in the present invention which facilitates the bead to stand stationary and the cells grow over and around the bead and a complete nacre layer formed.

We claim:

1. An in vitro process for the production of pearl, said process comprising the steps:
a) obtaining pre-treated mantle strip of pre-determined size from an organism;
b) attaching the mantle strip of step (a) with shell bead nucleus and placing the same in nutrient rich medium;
c) allowing the mantle strip to grow over shell bead nucleus so as to form pearl sac;
d) culturing pearl sac of step (c) in nutrient rich medium to produce organic matrix;
e) inducing the cells of mantle strip by the organic matrix of step (d) to produce nuclei;
f) allowing the nacre to get deposited on nuclei of step (e) to obtain pearl;

2. A process as claimed in claim 1, organism is marine organism.

3. A process as claimed in claim 1, wherein pre-treatment is carried out by antibiotic selected from group comprising streptomycin and penicillin.

4. A process as claimed in claim 1, wherein predetermined size of pre-treated mantle strip lies in the range of 1 to 3 square mm.

5. A process as claimed in claim 1, wherein the nutrient rich medium comprising of:
(a) 0.8 gm agar powder dissolved in 57.2 ml triple distilled water
(b) Supplementary salt solution consisting of

| | | |
|---|---|---|
| NaCl | 102.4 | g/l |
| KCl | 1.8 | g/l |
| CaCl₂ | 1 | g/l |
| MgCl₂ | 8 | g/l |
| MgSO₄ | 16.7 | g/l |
| M 199 10x | 2 | ml |
| FCS | 10 | ml |
| Lactalbumin | 5 | ml |
| hydrolysate(10%) | | |
| NaHCO₃ (5%) | 0.7 | ml |
| Kanamycin(0.1 g/ml) | 1 | ml |

5. A process as claimed in claim 1, wherein in step (f), nacre is deposited within 15 days of initiation of cultures.

6. A process as claimed in claim 1, wherein the predetermined colour is imparted to pearls.

7. A process as claimed in claim 1, wherein the predetermined shape is imparted to pearls.

8. A process as claimed in claim 1, wherein size of the pearl is controlled by using shell bead nucleus in the range of 2 mm to 8 mm as claimed in claim 1 in step (b).

9. A process as claimed in claim 1, wherein organism is selected from a group of sea molluscs, mussle and snail.

10. A process as claimed in claim 10, wherein the organism is Pinctada fucata and Haliotis varia.
